(19) United States
(12) Patent Application Publication
(10) Pub. No.: US 2007/0265608 A1
(43) Pub. Date: Nov. 15, 2007

(54) Method of energy ablation for the treatment of gastrointestinal diseases

(76) Inventor: Lyndon V. Hernandez, Franklin, WI (US)

Correspondence Address: QUARLES & BRADY LLP, 411 E. WISCONSIN AVENUE, SUITE 2040, MILWAUKEE, WI, 53202-4497, US

(21) Appl. No.: 11/656,192

(22) Filed: Jan. 22, 2007

Related U.S. Application Data
(60) Provisional application No. 60/761,408, filed on Jan. 23, 2006.

(51) Int. Cl. A61B 18/02

(52) U.S. Cl. 606/21

(57) ABSTRACT

A closed tip cryoprobe is advanced transorally or transrectally through the accessory channel of an endoscope to a region of interest in the gastrointestinal tract. In one embodiment, the cryoprobe is positioned near the gastric pacemaker region in the stomach and provides cooling of the region for a period of several minutes in order to increase the gastric emptying period.

### CROSS REFERENCE TO RELATED APPLICATIONS

This application claims priority to U.S. provisional patent application Ser. No. 60-761408, filed Jan. 23, 2006, and titled “Method for Cryotherapy of Gastrointestinal Diseases,” which is hereby incorporated by reference.

### FIELD OF THE INVENTION

The invention relates to methods for using an ablative energy source, such as a closed tip cryoprobe, for the treatment of various conditions and diseases such as obesity, Barrett's esophagus, tumors of the gastrointestinal (GI) tract, gastrointestinal reflux disease (GERD), and fecal incontinence.

### BACKGROUND OF THE INVENTION

There are several conditions and diseases relating to the gastrointestinal tract that pose technical challenges to the physician. For example, obesity, as defined by the World Health Organization, affects over one billion adults globally, and represents a risk factor for several chronic diseases that lead to premature death. Gastric bypass surgery is the gold standard treatment for patients with a body mass index of over 27 and other health complications related to obesity. Although this type of surgery has a success rate of up to 65%, several complications can develop, including nutritional deficiencies, stomal ulcer, and anastomotic stricture. Further, this is an expensive procedure. A need exists for a less invasive and less expensive procedure that prolongs the sensation of satiety in obese patients.

Gastric emptying appears to be more rapid among obese individuals compared to lean individuals, which may explain why satiety levels of obese people remain low soon after ingesting a high-calorie meal. Although it is difficult to compare gastric emptying between published studies because the method of measuring gastric emptying is not standardized at the present time, gastric emptying appears to be a factor that influences satiety levels which in turn affects food intake and body weight.

The muscle in the gastrointestinal tract is made up of smooth type of muscle. Within the wall of the gastrointestinal tract is the enteric nervous system that is a network of nerves involved with the process of digestion such as motility, secretion, and absorption. The enteric nerves are organized into interconnected networks called plexuses. Of these, the myenteric plexus, situated between the circular and longitudinal smooth muscle layers, is the main modulator of gastrointestinal motility.

Attempts have been made to decrease gastric emptying rate to induce satiety that would lead to weight loss. One approach involves the alteration of the motor and sensory functions of the stomach. Injection of botulinum toxin, an inhibitor of acetylcholine-mediated gastric peristalsis, into the gastric antrum could lead to some degree of weight reduction. However, botulinum toxin injection does not appear to have a significant, sustainable effect in weight loss.

Another disease of concern to the physician is GERD which is often a chronic condition that requires long-term acid suppressing medication for many patients. Some of these patients may be candidates for non-medical treatment, specifically endoscopically-guided radio frequency energy (RFE) directed into the lower esophageal sphincter to create small thermal lesions, which later heal and contract the sphincter muscle. Although this procedure has been shown to improve GERD symptoms, up to 70% of these patients will need to resume their medication within 5 years.

A similar lack of effective treatment exists for fecal incontinence. Although studies of RFE delivery to the anal canal have shown a median 70% resolution of symptoms, no long-term data exists. In addition, RFE technology is not readily available throughout the nation.

Barrett's esophagus, a complication of GERD, is a condition where the normal lining of the esophagus is replaced by a pre-cancerous columnar type of epithelium. Photodynamic therapy (PDT) is currently the only Food and Drug Administration (FDA) approved modality for ablating Barrett's esophagus. However, PDT can result in esophageal stricture in up to 40% of patients, photosensitivity, as well as chest pain. Additionally, the lifetime cost of PDT is close to $50,000, which is twice the lifetime cost for an esophagectomy.

For patients with tumors of the gastrointestinal tract (such as esophageal cancer), laser is the preferred treatment option, but this is an expensive procedure that is typically not readily available to community gastroenterologists.

In general, cryosurgical probes are known and are used to treat a variety of diseases. For example, cryosurgical probes are used to quickly freeze diseased body tissue, causing the tissue to die after which it either will be absorbed by the body, expelled by the body, sloughed off or replaced by scar tissue. Cryothermal treatment is currently used to treat prostate cancer and benign prostate disease, breast tumors including breast cancer, liver tumors including cancer, glaucoma and other eye diseases. Cryosurgery may also be used for the treatment of a number of other diseases and conditions including the treatment of cardiac arrhythmias, such as atrial fibrillation.

A variety of cryosurgical instruments, variously referred to as cryoprobes, cryosurgical probes, cryosurgical ablation devices, and cryostats and cryocoolers, are available for cryosurgery. These devices typically use the principle of Joule-Thomson expansion to generate cooling. They take advantage of the fact that most fluids, when rapidly expanded, become extremely cold. In these devices, a high pressure gas such as argon or nitrogen is expanded through a nozzle inside a small cylindrical shaft or sheath typically made of steel, and the Joule-Thomson expansion cools the steel sheath to a cold temperature very rapidly.

Open-tipped cryoprobes have been described for use in the GI tract, involving liquid or gaseous cryogen spray delivered by feeding the cryoprobe into the accessory channel of an endoscope. The cryogen is then sprayed at a distance from the target lesion. The depth of injury induced by an open-tipped cryoprobe (with a catheter-tip pressure of 2 to 4 psi), is typically approximately 2 mm [Johnston M H et al, Gastrointest Endosc. 2005 December;62(6):842-8]. However, these open-tipped cryoprobes emit gas into the intestinal lumen, which may necessitate the concurrent use of nasogastric suction that adds to patient discomfort.

### SUMMARY OF THE INVENTION

One aspect of the invention relates to the treatment of obesity using an ablative energy source. In one embodiment, a probe such as a cryoprobe is positioned near a region of interest in the stomach, such as the gastric pacemaker region, and used to deliver ablative energy with the goal of altering the motor and sensory function and leading to weight reduction. Various other types of ablative energy can also be used in the treatment of obesity. Ablative energy sources discussed herein include, but are not limited to, radiofrequency, cryoenergy, microwave, laser, and high intensity ultrasound. The ablative energy such as the cyro energy applied by a cryoprobe can alter the physical characteristics of the gastrointestinal tract by producing varying depths of injury from the internal surface of the gastrointestinal tract. The sensory and motor nervous system of the gastrointestinal tract resides in the subepithelial layer, which is within the range of the ablative energy.

Another aspect of the invention is the recognition that the use of a closed tip cryoprobe in the gastrointestinal tract is advantageous as it does not generate gas at its tip as it returns the cryogen (typically nitrous oxide vapor) via suction at the tip of the probe. This design eliminates the need to suction insufflated gas from the intestinal lumen, thus making a cryotherapy technique in the gastrointestinal tract less complicated. A closed tip cryoprobe also has the ability to direct therapy more precisely, as it involves contact between the cryoprobe and the target lesion or region of interest. This type of cryoprobe has been used in clinical practice for the endovascular treatment of cardiac arrhythmias, and is currently available in many tertiary medical centers across the United States. The closed tip cryoprobe is designed for use inside a blood vessel. However, the GI tract has heat loads that are much less than in a flowing blood environment. In absolute terms the closed tip cryoprobe will be more powerful because the refrigerant is typically pure liquid N₂O and so more energy will be extracted from the liquid to gas phase change (per unit volume of refrigerant) than with a gas system (such as Erbe) where energy extracted will be from the gas expansion in a so called Joule-Thomson expansion. A more complex and powerful cryotherapy system is required in the gastrointestinal tract in specific disease states in order to efficiently provide a prolonged effect on the nerve cells in the GI tract.

Therefore, these closed tip cryoprobes are ideal for the five specific GI applications mentioned above that currently have no effective treatment, namely for the treatment of obesity, Barrett's esophagus, tumors of the gastrointestinal tract, GERD, and fecal incontinence.

### BRIEF DESCRIPTION OF THE DRAWING

FIG. 1 is a schematic illustration of a cryoprobe in the accessory channel of an endoscope which is positioned in the stomach.

### DETAILED DESCRIPTION OF THE INVENTION

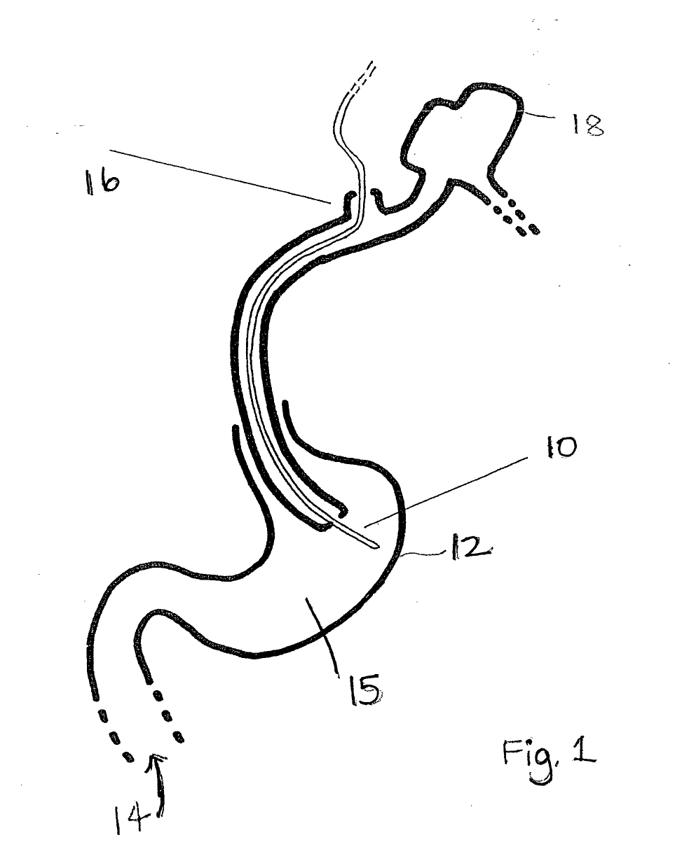

The invention relates to a method for treating obesity by increasing satiety levels using ablative energy and for treating other gastrointestinal diseases with cryotherapy using a closed tip cryoprobe. As illustrated in FIG. 1, a cryoprobe 10 or other energy delivery probe is positioned near a region of interest in the gastrointestinal tract 14. For example, the probe can be inserted in the accessory channel 16 of an endoscope 18, with its tip located inside the gastrointestinal tract. Preferably adequate steps are taken to assure safe and comfortable passage of this instrument inside the GI tract 14 or abdominal cavity 15. These steps may include pre-procedure fasting, bowel preparation, and/or appropriate sedation as clinically indicated.

The probe 10 can be introduced from an exterior of a subject through any natural orifice such as the mouth, nose, anus, or through an artificially made opening through the skin. The probe 10 may be introduced with or without the aid of direct or indirect visualization, including but not limited to endoscopes and laparoscopes. The objective is to cause temporary or permanent physical changes within the gastrointestinal tract, leading to the intended effect for treating a specific gastrointestinal disease as more fully explained below.

Once the cryoprobe 10 or other energy delivery probe is positioned near a region of interest in the GI tract, energy is applied for a predetermined amount of time to the region of interest. When using a cryoprobe and associated cryosurgical system, the region of interest is cooled. The method of the present invention can be performed using equipment such as is disclosed in U.S. Pat. Nos. 6,546,932, 6,772,766, 6,736,809, 6,648,880, 6,475,212, 6,936,045, and 5,846,235 for example.

The probe can be used with an endoscope to facilitate visualization by inserting it into the accessory channel of the endoscope (including but not limited to esophagogastroduodenoscope or EGD, flexible sigmoidscope, colonoscope, echoendoscope or endoscopic ultrasound, and side-viewing duodenoscope for endoscopic retrograde cholangiopancreatography or ERCP), or passed along side an endoscope as dictated by the clinical indication. When a probe is inserted into the accessory channel of an endoscope, the probe should have a flexible member with an outer diameter ideally of no more than 10 mm in order to facilitate insertion into the accessory channel of an endoscope. A probe can also be introduced into a subject without the use of an endoscope, or it can also be introduced during, before, or after laparoscopy.

When delivering cryoenergy, a cryoprobe is connected to an electromechanical control console that processes and delivers a refrigerant distally towards the tip of the catheter. The cryoprobe may have a single lumen, or more than one lumen. A dual lumen cryoprobe comprises a chamber that conducts the liquid or gas cryogen distally and another lumen with constant vacuum that recovers the refrigerant gas generated. The depth of tissue injury will preferably be in the range of 0.1 mm to 20 mm, depending on the amount and time the tissue is exposed to the cryo energy, as well as the mechanical properties of the specific cryoprobe or RFA used. The depth of tissue injury can be varied from this range.

The method of cryotherapy varies according to the following clinical conditions:

For the treatment of Barrett's esophagus, preferably the endoscope is passed transorally into the esophagus, and the closed tip cryoprobe is advanced with or without video guidance into the Barrett's mucosa. After the cryoprobe tip is maintained in position on the Barrett's mucosa, cryo energy is applied. This may be repeated in several sessions as necessary to ablate the columnar intestinal metaplasia. Chromoendoscopy may or may not be used along with this method to further evaluate the Barrett's esophagus.

For the treatment of tumors or cancerous lesions in the GI tract, including situations where curative resection is not possible, a step of dilating the lumen using a balloon or bougie may precede the cooling step to allow passage of the endoscope and/or closed tip cryoprobe at the level of the tumor. The endoscope can be inserted into the gastrointestinal lumen and the closed tip cryoprobe advanced into the area of tumor involvement. The area of interest may also include tumors of the liver and pancreatico-biliary tract (with the use of an echoendoscope or ERCP). Cryo energy is applied into either the surface or core of the tumor. To reach the core of the lesion, the cryoprobe should have an outer diameter small enough to slide into the lumen of a needle catheter, with the catheter being advanced into the surface or center of the lesion, and with the cryoprobe, a spherical cryo lesion is created at very low temperatures. Larger tumors may require more than a single treatment.

For the treatment of GERD and fecal incontinence, cryo energy is applied to the area of the lower esophageal sphincters (transorally for GERD), and to the internal and external anal sphincters (transrectally for fecal incontinence). This may or may not be performed using an endoscope. Cooling a region of interest using the closed tip cryoprobe which is passed transorally or transrectally alters the characteristics of the sphincter muscle. The ideal depth of injury for this application is less than 10 mm.

For the treatment of obesity, the cryoprobe or other energy delivery probe can be targeted to a specific area of the gastrointestinal tract, preferably the gastric pacemaker region 12 located in the proximal body of the stomach. The stomach, divided into fundus and corpus, is characterized by electrical phasic activity. The gastric pacemaker region is generally located at the proximal stomach along the greater curvature.

The cryoprobe or other energy delivery probe is operable to deliver ablative energy to induce the stomach to increase the duration of emptying of stomach contents, leading to increased satiety and subsequent weight loss. The probe can also be positioned near the pylorus, or cardia. The ablative energy is applied for a predetermined period of time. Preferably, a cryoprobe is transorally positioned near the gastric pacemaker region in the stomach and cryo energy is applied for a period of 2 minutes or so, and then the probe is removed. This non-implantable method of delivering ablative energy operates to prolong satiety by increasing the gastric emptying period.

Ablative energy sources for the treatment of obesity include radiofrequency, cryoenergy, microwave, laser, and high intensity ultrasound. Ablative energy is applied to the area of interest the gastrointestinal wall, which can be the inside the lumen of the gastrointestinal tract or outside through a transcutaneous route. The ablative energy alters the sensori-motor function of the gastrointestinal wall, and causing gastrointestinal contents to be slowly propelled distally.

The ablative energy is delivered by an energy delivery probe and directed into the target tissue of interest at a desired region of interest in the gastrointestinal tract. The energy delivery probe can take various configurations and be positioned near an internal or external surface along the entire gastrointestinal tract. This probe can be introduced from an exterior of a subject through a natural orifice such as the mouth, nose, anus, or through an artificially made opening through the skin. The probe may be introduced with or without the aid of direct or indirect visualization, including but not limited to endoscopes and laparoscopes.

The method steps described above can be repeated a number of times, and may be combined with systemic pharmacologic agents that synergistically produce the desired effect of inducing weight loss.

While the method of the invention was described using various embodiments, the invention is not to be limited to the disclosed embodiments, but rather intended to cover various modifications included within the scope of the claims.

What is claimed is:

1. A method for treating obesity by increasing satiety levels, the method comprising:
positioning a cryoprobe near a region of interest in the stomach; and
cooling the region of interest using the cryoprobe for a predetermined amount of time.

2. The method of claim 1, wherein the region of interest is selected from the group including the gastric pacemaker region, pylorus, and cardia.

3. The method of claim 1, wherein the predetermined amount of time is on the order of several minutes.

4. The method of claim 1, wherein the cryoprobe is a closed tip cryoprobe.

5. The method of claim 1, wherein the positioning step occurs with the aid of an endoscope.

6. The method of claim 1, wherein the positioning step occurs with the cryoprobe located in an accessory channel of an endoscope.

7. The method of claim 1, wherein the positioning step occurs either transorally or transrectally.

8. The method of claim 1, wherein the cryoprobe is located outside of the stomach during the cooling step.

9. The method of claim 1, wherein the cryoprobe is located inside the stomach during the cooling step.

10. A method for treating obesity by increasing satiety levels, the method comprising:
positioning an energy delivery probe near a region of interest in the stomach; and
delivering ablative energy to the region of interest using the probe for a predetermined amount of time to alter the sensori-motor function of the gastrointestinal wall to cause stomach content to be more slowly propelled distally therefrom.

11. The method of claim 10, wherein the region of interest is selected from the group including the gastric pacemaker region, pylorus, and cardia.

12. The method of claim 10, wherein the positioning step occurs with the aid of an endoscope.

13. The method of claim 10, wherein the positioning step occurs with the probe located in an accessory channel of an endoscope.

14. The method of claim 10, wherein the positioning step occurs either transorally or transrectally.

15. The method of claim 10, wherein the probe is located outside of the stomach during the cooling step.

16. The method of claim 10, wherein the probe is located inside the stomach during the cooling step.

17. A method for treating Barrett's esophagus, gastrointestinal, liver, or pancreatico-biliary tumors, gastrointestinal reflux disease, fecal incontinence, or obesity, comprising:
positioning a closed tip cryoprobe near an area of interest in the gastrointestinal tract, and
cooling the region of interest using the cryoprobe for a predetermined amount of time to produce a desired effect.

18. The method of claim 17 for treating Barrett's esophagus, wherein the cryoprobe operates to ablate the columnar intestinal metaplasia.

19. The method of claim 17 for treating tumors, wherein tumor tissue is frozen.

20. The method of claim 17 for treating gastrointestinal reflux disease, wherein the cooling step is performed to alter the characteristics of the sphincter muscle.
